(19) United States
(12) Patent Application Publication
(10) Pub. No.: US 2008/0045896 A1
(43) Pub. Date: Feb. 21, 2008

(54) Catheter having intermediate position markers

(75) Inventors: Travis R. Yribarren, San Mateo, CA (US); Randolf Von Oepen, Los Altos Hills, CA (US); Arik Zucker, Zurich (CH); Lorcan Coffey, Tubingen (DE); Thomas Rieth, Hirrlingen (DE)

Correspondence Address: BEYER WEAVER LLP/Abbott Vascular Devices, P.O. BOX 70250, OAKLAND, CA, 94612-0250, US

(73) Assignee: Abott Laboratories

(21) Appl. No.: 11/506,309

(22) Filed: Aug. 17, 2006

(51) Int. Cl. A61M 29/00; A61M 31/00; A61M 37/00

(52) U.S. Cl. 604/103.1

(57) ABSTRACT

Various improved catheter marking arrangements are described. In a first aspect of the invention, the catheter includes an expandable member (such as for example, a balloon, a stent, etc.). A pair of radiopaque end markers mark the distal and proximal ends of the expandable member. Additionally, a radiopaque intermediate marker is provided that marks an intermediate position on the expandable member. The intermediate marker may be used to mark important points on the expandable member. The markers are sized and positioned such that a surgeon utilizing the catheter in a surgical procedure would be able to readily differentiate the markers in a fluoroscopic image to determine the location of the item marked by the orientation of the catheter. In various other aspects of the invention, the intermediate marker is arranged to mark a position on the expandable member that constitutes a line of demarcation for an agent (such as a drug) carried by a balloon.

### BACKGROUND OF THE INVENTION

The present invention relates generally to radiopaque markers provided on catheters to help surgeons position and/or orient specific components of the catheters during use.

Catheters are used in a wide variety of medical procedures. Typically catheters are relatively long and flexible. Many times, (as for example in many vascular applications) a catheter is inserted into a relatively tortuous vessel. In order to better track the location of the catheter, it is common to image the catheter during its insertion, placement and/or use. Thus, the surgeon can see that the catheter is properly positioned in situ. Today, one of the most common surgical imaging techniques is fluoroscopy. Often, some of the components at the working end of the catheter (e.g., balloons, ports, etc.) may be difficult to identify using fluoroscopy in the midst of a surgical procedure. Accordingly, radiopaque markers have sometimes been used to mark specific points on a catheter. By way of example, in many balloon catheters used in common angioplasty procedures, markers may be positioned on the catheter at or about the distal and proximal ends of the inflatable balloon in order to mark the location of the balloon. Such an arrangement is illustrated in FIG. 4. Commonly, the markers are formed from simple metal bands that are crimped to the catheter, although a wide variety of different materials and assembly techniques have been used to create visible markers.

Although there are currently a number of catheters that have integrated radiopaque markers into their design, there is always a desire to improve the usability of devices used in surgical procedures. Accordingly, there are continuing efforts to provide improved marking arrangements that improve the usability of various catheters.

### SUMMARY OF THE INVENTION

To achieve the foregoing and other objects of the invention, various improved catheter marking arrangements are described. In a first aspect of the invention, the catheter includes an expandable member (such as for example, a balloon, a stent, etc.). A pair of radiopaque end markers mark the distal and proximal ends of the expandable member. Additionally, a radiopaque intermediate marker is provided that marks an intermediate position on the expandable member. The intermediate marker may be used to mark important points on the expandable member. The markers are sized and positioned such that a surgeon utilizing the catheter in a surgical procedure would be able to readily differentiate the markers in a fluoroscopic image to determine the location of the item marked by the orientation of the catheter.

In another aspect of the invention, the intermediate marker is arranged to mark an intermediate position on the expandable member that constitutes a line of demarcation for an agent (such as a drug) carried by a balloon. The intermediate marker is spaced apart from the ends of the balloon. By way of example, the agent may be a drug, a biological agent, a chemical agent, radiation or other diagnostic, therapeutic or monitoring agents. This type of intermediate marker is particularly useful in devices (such as balloon catheters) where an agent (such as a drug) is carried by a portion of the balloon, but not all of the balloon, and in devices where the concentration of the agents and/or the agent mix is varied over the face of the balloon. The described markers allow a surgeon utilizing the catheter to readily see the location of the line of demarcation. In some embodiments, multiple intermediate markers may be employed to mark other lines of demarcation on the expandable member.

The markers may be formed from any suitable materials that can be clearly detected by an imaging system. In some embodiments, the markers may be formed from metallic bands secured to the elongated flexible member. In other embodiments, the markers may be formed from radiopaque ink applied to either the balloon or the elongated flexible member.

The expandable member may take any suitable form including forms other than a balloon. For example, stents and various expansion devices may be used as the expandable member.

### BRIEF DESCRIPTION OF THE DRAWINGS

The invention, together with further objects and advantages thereof, may best be understood by reference to the following description taken in conjunction with the accompanying drawings in which:

FIG. 1 is a diagrammatic view of the distal end of a simplified catheter having a side port and rotational markers in accordance with one embodiment of the invention, with the distal end of the catheter positioned near a bifurcation in a vessel;

FIG. 2 is a cross sectional view of the catheter illustrated in FIG. 1 taken along line 2-2 of FIG. 1;

FIG. 3 is a diagrammatic view of the distal end of a catheter having rotational markers in accordance with another embodiment of the invention;

FIG. 4 is a diagrammatic view of the distal end of an angioplasty catheter utilizing a conventional balloon marker arrangement;

FIG. 5 is a diagrammatic view of the distal end of a catheter utilizing a marker arrangement that includes an intermediate marker in accordance with one embodiment of the invention;

FIG. 6 is a diagrammatic view of the distal end of a catheter utilizing a marker arrangement that includes an intermediate marker in accordance with another embodiment of the invention.

It is to be understood that, in the drawings, like reference numerals designate like structural elements. Also, it is understood that the depictions in the figures are diagrammatic and not to scale.

### DETAILED DESCRIPTION OF THE PREFERRED EMBODIMENTS

The present invention relates generally to specific marker configurations that are suited for use with specific types of catheters.

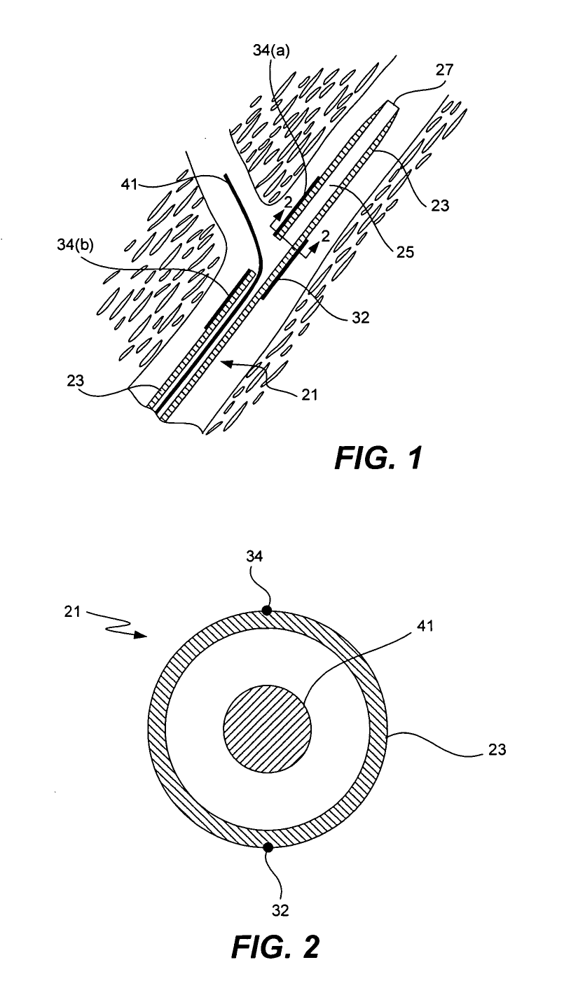

Referring initially to FIG. 1, a marker arrangement arranged to mark the rotational orientation of the distal end of a catheter in accordance with one embodiment of the invention will be described. The catheter 21 is generally formed from an elongated tubular member 23 having central lumen 25 formed therein. The central lumen opens at the distal end of the catheter at a distal port 27 such that the catheter can be inserted into a target vessel over a guide wire 41. The catheter also has a side port 29 that opens to the side of the catheter. This type of side port can be useful in a variety of different catheter designs, as for example in certain bifurcation catheters, atherectomy catheters, perfusion catheters, and support catheter designs.

A first longitudinally extending marker 32 is positioned on the side of the catheter opposite the side port 29. A pair of longitudinally extending axially aligned markers 34(a), 34(b) are positioned on opposite (distal and proximal) sides of the side port 29. All of the markers are arranged to extend substantially parallel to the longitudinal axis of the catheter.

With the described arrangement a surgeon utilizing the catheter can readily identify both the axial location of the side port 29 and its rotational orientation. Specifically, the port is bounded by a pair of axially aligned markers 34(a), 34(b) that together constitute segments of a “long” marker, thus making it easy to identify the location of the port. The side port 29 is also effectively marked by the marker 32, which is located opposite the side port. Marker 32 is shorter than the combined end-to-end length of the axially aligned markers 34. Therefore, the markers can readily be distinguished on a fluoroscopic image when the catheter is in use. It should be appreciated that the length of each “segment” of the “long” marker may be the same length, shorter or longer than the “short” marker. Of course the respective lengths of the markers could be reversed as well.

A wide variety of materials may be used to form the markers. What is important is that the markers can be seen and identified during use of the catheter by standard fluoroscopes and/or other imaging devices that are expected to be used to track the catheter during a surgical procedure. By way of example, a variety of bio-compatible metals such as platinum, gold, tantalum, and stainless steel work well. So long as the markers are not also being used as electrodes, radiopaque inks or other non-conductive materials that can readily be imaged may be used as well.

The length and width of the markers may vary widely based on the needs of a particular system. What is important is that the markers can be readily observed and distinguished during a surgical procedure. By way of example, in many applications, the width of the markers would be less than approximately 0.005 inches, and preferably less than 0.003 inches, although this is not a requirement. Generally, end to end lengths in the range of between about 5 and 40 mm are preferred.

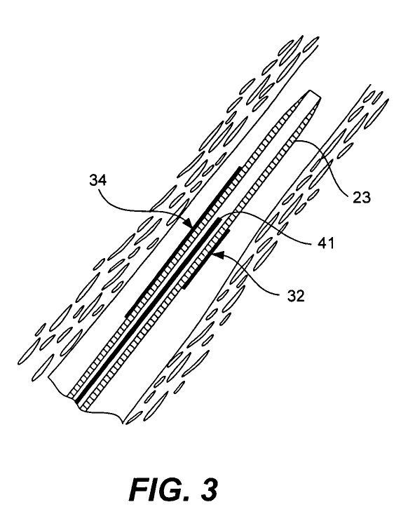

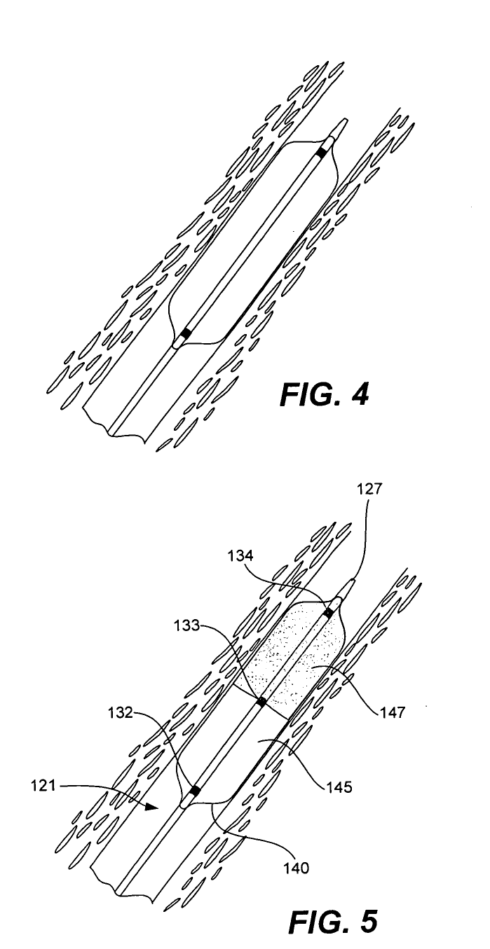

The embodiment illustrated in FIG. 1 utilizes the rotational markers to mark the location of a side port in a bifurcation catheter. It should be appreciated that the described rotational marker arrangement can be used to mark the rotational alignment of a wide variety of other types of catheters in which it is desirable for the surgeon to know the rotational alignment of the catheter. For example, as treatment plans become more sophisticated, it is believed that it may become more desirable to mark the rotational orientation of a catheter in various drug delivery, stent delivery, biopsy and angioplasty catheters. In embodiments that do not include a side port as illustrated in FIG. 1, the “long” marker can take the form of a single marker that is substantially longer than the “short” marker. Such an arrangement is illustrated in FIG. 3. It should be appreciated that by using markers of different lengths, it is easier for the surgeon to readily distinguish the markers and thus the catheter's rotational orientation during use.

The markers do not need to be continuous. Thus, in other embodiments, the markers may be formed from a series of dots, dashes or segments. In still other embodiments, the markers may be distinguished by characteristics other than, or in addition to, length. For example, one marker may be formed by a series of dashes while the second marker is a solid line, a series of dots, a series of dashes of different size or a different pattern of dots or dashes. In another example, the distance between dots, dashes or segments may be a distinguishing characteristic.

The markers can be formed using a variety of procedures. By way of example, one suitable method for forming the markers utilizes short strand of metal wire to form the markers. In one example, the wire is embedded in the catheter by forming a slit in the outer surface of the catheter, inserting the wire strand in the slit and sealing the slit. In another example, the wire may be attached to the outer surface of the catheter using a bio-compatible adhesive. In still other applications, metallic markers may be ion implanted onto the surface of the catheter. In still other applications, radiopaque inks can be printed onto the surface of the catheter in order to form the markers. In still other applications, the markers may be thermally bonded into the catheter. Of course a variety of other techniques could be used to form the markers as well.

Referring next to FIG. 5 a marker arrangement in accordance with another aspect of the invention will be described. In this embodiment, a series of three markers are provided on a balloon catheter 121. The catheter 121 is generally formed from an elongated tubular member 123 having a plurality of lumens formed therein. These may include a central lumen (not shown) that opens at the distal end of the catheter at a distal port 127 such that the catheter can be inserted into a target vessel over a guide wire. The catheter has an inflatable angioplasty balloon 140 that is secured to the periphery of the catheter near its distal ends. The elongated tubular member 123 has an inflation lumen that opens in a region bounded by the balloon 140 to facilitate inflation of the balloon during use.

A series of three longitudinally spaced marker 132, 133 and 134 are positioned on the catheter. The proximal marker 132 marks the proximal end of the balloon 140, the intermediate marker 133 marks an intermediate location on the balloon and the distal marker 134 marks the distal end of the balloon. As discussed in the background section, there are a variety of balloon catheters in use today that utilize markers to mark the proximal and distal ends of a balloon. However, we are unaware of balloon catheters that also utilize intermediate markers 133 to mark intermediate locations within a balloon. Such markers have a variety of uses. For example, in one application, the middle marker may be used to indicate the position of the stent relative to the ostium of a bifurcation. In another application, some medical procedures contemplate delivering drugs, biological agents, chemical agents, radiation or other materials using a balloon catheter. For simplicity, these various materials are generically referred to as “agents” herein. For example, drug eluting balloons and drug eluting stents are commonly used to deliver drugs to a vascular region under treatment. Today, when a balloon or a stent is designed to deliver an active agent, the entire surface (or the majority of the surface) of the balloon/stent is typically coated with the agent. However, it is believed that in some procedures, it may be desirable to treat only a portion of the surface of the balloon/stent so that the agent is delivered to a shorter region of the vessel than is treated by the balloon/stent. That is, only a portion of the balloon might be coated with an agent and the intermediate marker might be used to mark the location of the agent. For example, in a catheter where the distal portion 147 of the balloon is coated with an agent and the proximal portion 145 is not, the middle marker may be used to demarcate the coated from the uncoated regions of the balloon. Such an arrangement is shown in FIG. 5. Specifically, the proximal marker 132 marks the proximal end of the balloon 140, the intermediate marker 133 marks a line of demarcation where the agent begins to coat the balloon (i.e., the proximal end of the agent). The distal marker 134 marks the distal end of the balloon (which is the distal end of the coated region as well).

In another example, when treating a branched region of a vascular vessel, it may be desirable to stent the main branch of an artery and to perform an angioplasty operation in the side branch using a drug-eluting balloon to both deploy the stent and perform the angioplasty operation. In such a case it may be desirable to coat a distal portion of the balloon with a higher dosage of the agent than the proximal portion of the balloon so that the region of the vessel upstream of the branch is not exposed to a double dosage of the agent. In such an arrangement, the catheter may have a set of three markers. The distal and proximal markers mark the distal and proximal ends of the balloon (or the beginning and end of the agent if desired). The middle marker may then be arranged to identify the point where the concentration of the agent changes. That is, a line of demarcation for the agent.

In the embodiments described above, a single middle marker is provided. However, it should be appreciated that in a variety of applications it may be desirable to provide multiple middle markers. For example, if a central region of the balloon is coated with an agent while the proximal and distal ends are not, then a pair of middle markers may be used to mark the distal and proximal boundaries of the agent. Of course other suitable markers could be provided to mark other significant boundaries as well and thus, the number of middle markers that are used may be varied to meet the needs of particular applications.

It should be appreciated that there are potentially a wide variety of applications where it might be desirable to deliver agents to a region that is less than the entire length of a drug delivery balloon (or stent) or to vary the dosage of the agent along the length of the balloon (or stent). The described intermediate markers can be used to mark the beginning or ending boundaries of an agent, lines of demarcation between different agent concentrations or between different agents or agents mixes that are employed on a single balloon or stent.

In the previously described embodiments, the markers were placed on (or formed in) the catheter itself as opposed to on a balloon, stent or other item that is carried by the catheter. However, in other embodiments, suitable radiopaque markers may be made directly on a balloon (or other component). By way of example, radiopaque inks are particularly well suited for forming intermediate markers directly on a balloon.

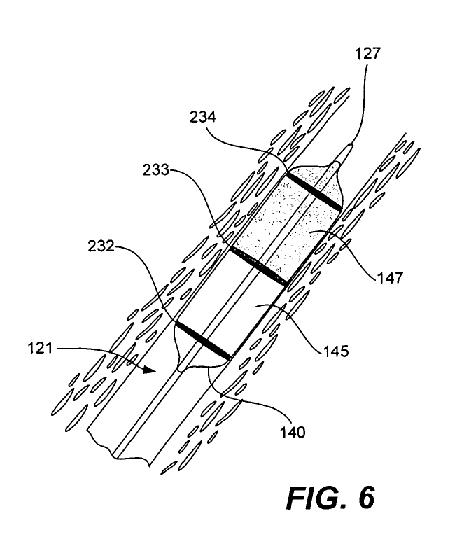

Just as in the previously described embodiment, the markers may be formed using any suitable technique. By way of example, metal bands may be secured to the catheter using standard crimping techniques to form the markers. Alternatively metal wires may be used to form the marker. In other applications, thermal embedding or adhesives may be used to place the markers. In still other applications the markers may be created by ion implanted or printed suitable radiopaque materials onto the catheter or the balloon as discussed above. By way of example, FIG. 6 illustrates radiopaque markers 232, 233 and 234 that are printed on the balloon itself. Of course a variety of other techniques could be used to form the markers as well.

In some applications it may be desirable to use both the described rotational markers and the described intermediate markers on the same device or on a pair of devices that are intended to be used in conjunction with one another. For example, in a bifurcation catheter, it may be desirable to mark the side port with the described rotational marker and an angioplasty device that is used in conjunction with the bifurcation catheter may use the described intermediate markers.

The intermediate markers can also be used to mark certain other items on a balloon catheter. For example, a pair of markers could be used to mark the longitudinal location of a side port on the catheter.

The described markers can readily be distinguished on a fluoroscopic image when the catheter is in use. Therefore, a surgeon utilizing the described catheter can readily identify both (a) the location of a balloon (or stent or other expandable member), and (b) the segment of the balloon that is coated with an active agent. Specifically, the proximal and distal markers 132, 134 mark the proximal and distal ends of the balloon, while the intermediate marker 133 marks a line of demarcation for the agent applied to the balloon and thus the location of agent elution.

Although only a few embodiments of the invention have been described in detail, it should be appreciated that the invention may be implemented in many other forms without departing from the spirit or scope of the invention. Most notably, FIGS. 1 and 2 illustrate simplified catheter arrangements. It should be appreciated that in many applications, the catheters will have more sophisticated designs that are specific for their intended tasks. For example, many catheters employ multi-lumen tubular members. These might include separate balloon inflation lumens, access lumens etc.

A few specific applications have been described. For example, the rotational markers have been primarily described in the context of marking a side port. However, it should be appreciated that the described markers may be utilized in a wide variety of other applications including biopsy catheters, atherectomy catheters, perfusion catheters, support catheters, etc.

Similarly, the longitudinal marker arrangement has been described primarily in the context of drug (agent) eluting balloon catheters. However, again, they may be arranged to mark other important features on a balloon (or other expandable member) as well. Therefore, the present embodiments are to be considered as illustrative and not restrictive and the invention is not to be limited to the details given herein, but may be modified within the scope and equivalents of the appended claims.

What is claimed is:

1. A catheter comprising:
an elongated flexible member suitable for insertion into a body vessel, the elongated flexible member having at least one lumen therein;
an expandable member carried by the elongated flexible member; and
a plurality of marker including a pair of end markers that mark the location of distal and proximal ends of the expandable member and an intermediate marker that marks an intermediate position on the expandable member.

2. A catheter as recited in claim 1 wherein the expandable member is a balloon.

3. A catheter as recited in claim 1 wherein the markers are metallic bands secured to the elongated flexible member.

4. A catheter as recited in claim 2 wherein the markers are formed from radiopaque ink applied to one of the balloon and the elongated flexible member.

5. A catheter as recited in claim 1 wherein the expandable member is a stent secured near a distal end of the flexible member, the catheter further comprising a stent delivery mechanism arranged to expand the stent such that the stent may be delivered to a desired location within a vessel.

6. A catheter comprising:
an elongated flexible member suitable for insertion into a body vessel, the elongated flexible member having at least one lumen therein;
an expandable balloon carried by the elongated flexible member, wherein the balloon carries an agent suitable for use in a medical procedure or a diagnostic procedure; and
a plurality of markers including at least one end marker that marks the location of a first end of the balloon and an intermediate marker that marks an intermediate position on the balloon that constitutes a line of demarcation for the agent carried by the balloon that is spaced apart from a second end of the balloon.

7. A catheter as recited in claim 6 further comprising a second end marker that marks a second end of the balloon.

8. A catheter as recited in claim 6 further comprising a second intermediate marker that is spaced apart from the other markers and marks a second line of demarcation for the agent or agents carried by the balloon.

9. A catheter as recited in claim 6 wherein the agent is selected from the group consisting of drugs, biological agents, chemical agents and radiation.

10. A catheter as recited in claim 6 wherein the agent is a diagnostic agent.
